(19) United States
(12) Patent Application Publication
(10) Pub. No.: US 2016/0287614 A1
(43) Pub. Date: Oct. 6, 2016

(54) Stable Pharmaceutical Formulation(s) of Tetracycline Antibiotic

(71) Applicant: LUPIN LIMITED, Mumbai (IN)

(72) Inventors: Amol Mandhare, Pune (IN); Nilendu Sen, Thane (W) (IN); Paul B. McGarty, Baltimore, MD (US)

(21) Appl. No.: 15/037,822

(22) PCT Filed: Nov. 19, 2014
(86) PCT No.: PCT/IB2014/066152

§ 371 Date: May 19, 2016

(30) Foreign Application Priority Data
Nov. 20, 2013 (IN) 3646/MUM/2013

(51) Int. Cl. A61K 31/65; A61K 8/35; A61Q 17/04; A61K 9/00

(52) U.S. Cl. CPC A61K 31/65; A61K 9/0014; A61K 8/35; A61Q 17/04

(57) ABSTRACT

The present invention provides a stable, organoleptically acceptable topical formulation(s) of the tetracycline antibiotics or pharmaceutically acceptable salts or hydrates thereof for the treatment of acne.

### BACKGROUND OF THE INVENTION

Acne is a common inflammatory disease in skin areas where sebaceous glands are largest, most numerous and most active. In its mildest form, it is a more or less superficial disorder which is evidenced by slight, spotty skin irritations and ordinary skin hygiene is a satisfactory treatment. However, in the more inflammatory types of acne, bacterial invasion of or about the pilosebaceous follicle occurs and pustules, infected cysts, and in extreme cases canalizing inflamed and infected sacs appear. Without effective treatment, these lesions may become extensive and leave permanent, disfiguring scars.

Acne is very common in puberty. As reported by Hunnitz, S.: Clinical Pediatric Dermatology, p. 107 Philadelphia, W. B. Saunders Co., 1981, up to 85 percent of high school students have acne lesions and it is realistic to say that acne is so common 100 percent of persons between 9 and 19 have some experience with acne lesions. Usually by the early twenties the process of lesion formation slows considerably.

While acne is not a life-threatening disease, it may be cosmetically and emotionally disabling. The facial eruptions are known to cause psychic trauma. The sufferer may be constantly aware of the obvious facial blemishes. Thus, the immediate goals of treatment are to limit the physical and psychological scarring.

The etiology of lesion formation is viewed in the following way. The earliest acne lesions are comedones and are the result of the failure to normally slough the horny epidermal cells lining the follicular canal. Dilation of the orifice of a sebaceous follicle above a comedo will result in the extrusion of this mass at the surface resulting in an open comedo, a blackhead. If the pore above a comedo fails to dilate, then an impaction becomes a closed comedo or whitehead. The formation of a closed comedo can be followed by inflammatory lesions. Papules, pastules, modules and cysts may result from a process in which an impacted follicular sebaceous unit becomes the site of action of several products of Corynebacterium acnes, (P. Acnes) normal anaerobic bacteria. Treatment of acne by a physician then becomes necessary.

Treatments that are currently used to treat acne include comedolytics, exfoliants, oral and topical bacteriostatics as well as systemic antibiotics.

It is well known that the tetracycline antibiotics, and especially minocycline hydrochloride, are particularly effective in treating the condition when administered systemically. Minocycline base is easily transportable through the epidermis, due to its high solubility in lipids, whereas minocycline in the form of its acid addition salts (e.g. hydrochloride), is more difficult to transport to the place of action, is acidic and is more aggressive for the skin.

However, oral antibiotics can cause candidial vaginitis, photoreaction, onychlysis and gram-negative folliculitis, as well as headaches, dizziness and other central nervous system side effects.

Topical antibiotics offer the advantage of a decreased total absorption of the drug and an accompanying decrease in toxicity as compared with systemic antibiotics. Additionally, topical antibiotics offer the added benefit of applying the medication solely to the targeted lesions.

Tetracyclines have limited stability in aqueous solutions (A. Kubis et al., “Investigation of stability of tetracycline hydrochloride in methylcellulose gel”, Pharmazie 42:519-520(1987)). Tetracycline antibiotics are known to be oxidatively unstable and often change from yellow to brown over time (Y. Liang et al., “Stability studies of tetracycline in methanol solution”, J. Chromatography 827:45-55(1998)). Despite this, efforts have been made to formulate tetracycline compositions for topical administration. These efforts have been hindered, however, by the instability of the tetracycline compositions in the presence of water and other protic liquids. Tetracycline formulations in presence of water and other protic liquids, typically form various degradation products such as, but not limited to, epitetracycline, anhydrotetracycline, and epianhydrotetracycline which leads to a limited, commercially undesirable shelf life for such tetracycline products in aqueous media.

The tetracycline antibiotics have been incorporated into various nonaqueous vehicles. Tetracycline antibiotics in alcohol based solvents are disclosed in U.S. Pat. Nos. 3,219,529, 3,389,174 and 4,376,118.

However, the use of such alcohol based solvents has not been cosmetically acceptable due to irritation and drying of the skin. An aqueous ethanolic solution of tetracycline hydrochloride in combination with an equilibrium concentration of the degradation product 4-epitetracycline hydrochloride has been commercially marketed under the trade name “Topicycline”. However it is relatively unstable in its solution form due to continuous degradation.

The tetracycline antibiotics have also been formulated in nonaqueous ointment bases, which are stable over a long period of time. While such formulations are desirable in that they are occlusive and they provide better penetration of the drug to the active site than a solution, but their greasy consistency is particularly unacceptable in the treatment of acne.

WO 2010/149980 discloses a topical formulation comprising a tetracycline characterized in that the formulation comprises two separate parts: (i) a first part comprising a tetracycline in solid form suspended in a first vehicle; and (ii) a second part comprising a second vehicle in which the tetracycline is soluble.

WO 2008/097851 discloses a tetracycline formulation for topical administration comprising at least one tetracycline or a pharmaceutically acceptable salt or hydrate thereof substantially stabilized in a base, wherein the base comprises at least one hydrophobic, non-hygroscopic silicone thickening agent and wherein the formulation is substantially free of protic liquids (including water).

WO 2008/097850 concerns multi-part tetracycline formulations for topical administration, as well as to methods of making and administering the same.

Thus, there is a need for a topical formulation(s) of the tetracycline antibiotics or pharmaceutically acceptable salts or hydrates thereof for the treatment of acne which is stable, provides good delivery of the drug to the skin surface, and yet is cosmetically acceptable for the purpose of acne therapy.

### SUMMARY OF THE INVENTION

In accordance, an object of the present invention is to provide a topical formulation comprising at least one tetracycline antibiotic or a pharmaceutically acceptable salt or hydrate thereof and a pharmaceutically acceptable base for the treatment of acne, wherein the tetracycline antibiotic is in stabilized form.

Yet another object of the present invention is to provide a topical formulation comprising at least one tetracycline antibiotic or a pharmaceutically acceptable salt or hydrate thereof and a pharmaceutically acceptable base for the treatment of acne, wherein the tetracycline antibiotic is in solubilized form.

Yet another object of the present invention is to provide a topical formulation comprising at least one tetracycline antibiotic or a pharmaceutically acceptable salt or hydrate thereof and a pharmaceutically acceptable base for the treatment of acne, wherein the tetracycline antibiotic is in suspended form.

Yet another object of the present invention is to provide a topical formulation comprising at least one tetracycline antibiotic or a pharmaceutically acceptable salt or hydrate thereof and a pharmaceutically acceptable base for the treatment of acne, wherein the tetracycline antibiotic is in solubilised form and optionally contains a sunscreen agent.

Yet another object of the present invention is to provide a topical formulation comprising at least one tetracycline antibiotic or a pharmaceutically acceptable salt or hydrate thereof and a pharmaceutically acceptable base for the treatment of acne, wherein the tetracycline antibiotic is in suspended form and optionally contains a sunscreen agent.

### DETAILED DESCRIPTION OF THE INVENTION

The present invention is directed to a topical formulation(s) of the tetracycline antibiotics or pharmaceutically acceptable salts or hydrates thereof in a stable form. In a preferred embodiment of the present invention, the topical formulation is suitable for external administration to the skin.

The term “topical” as employed herein relates to the use of the tetracycline antibiotic, incorporated in a suitable base, vehicle, or like carrier, and applied at the desired site for exertion of local action. Accordingly, topical administration includes those forms in which the medication is applied externally by direct contact with the skin surface to be treated. Conventional pharmaceutical forms for this purpose include ointments, lotions, pastes, creams, jellies, sprays, aerosols, bath oils and the like.

More preferred tetracyclines include, without limitation, tetracycline; 7-dimethylamino-6-deoxy-6-demethyltetracycline; 7-methylamino-6-deoxy-6-demethyl-tetracycline;9-methylamino-6-deoxydimethyl tetracycline; 7-ethylamino-6-deoxy-6-demethyltetracycline; 7-isopropylamino-6-deoxy-6-demethyltetracycline; 6-deoxy-5-hydroxytetracycline; oxytetracycline; 7-chlorotetracycline; 7-chloro-6-demethyltetracycline; 6-methyleneoxytetracycline; (2E,4S,4aR,5aS,12aR)-2-(amino-hydroxy-methylidene)-4,7-bis(dimethylamino)-10,11,12a-trihydroxy-4a,5,5a,6-tetrahydro-4H-tetracene-1,3,12-trione; (4S,4aR,5S,5aR,6R,12aS)-4-(dimethylamino)-3,5,10,12,12a-pentahydroxy-6-methyl-1,11-dioxo-1,4,4a,5,5a,6,11,12a-octahydrotetracene-2-carboxamide; tigecycline and the pharmaceutically acceptable salts and hydrates of the foregoing.

Specific examples of the most preferred tetracyclines include, without limitation, tetracycline, minocycline, doxycycline, oxytetracycline, chlortetracycline, demeclocycline, methacycline, tigecycline, and the pharmaceutically acceptable salts or hydrates of the foregoing. Special mention is made of minocycline and doxycycline, and their pharmaceutically acceptable salts or hydrates. Minocycline and its salts and hydrates are especially preferred for use in the present invention.

The term “protic liquid” refers to any liquid that carries a hydrogen attached to an oxygen (such as in a hydroxyl group), to a nitrogen (such as in an amine group) and further including any molecular liquid which contains dissociable H⁺. Examples of protic liquids include, but are not limited to, water, alcohols such as methanol, ethanol, glycerol, polyhydric alcohols and glycols such as ethylene glycol, propylene glycol, hexylene glycol and polyethylene glycol, acids such as acetic acid and formic acid, and bases such as ammonia.

As used herein, at least one tetracycline that is in “stable form” in a formulation refers to a formulation in which preferably more than about 85%, and more preferably more than about 90%, of the at least one tetracycline or its pharmaceutically acceptable salts or hydrates thereof remains after storage at 25° C. and 60% relative humidity (RH) for preferably about 3 months, more preferably about 6 months, and still more preferably about 12 months. “Stable form” can also refer to a formulation in which preferably more than about 85%, and more preferably more than about 90%, of the at least one tetracycline or its pharmaceutically acceptable salts or hydrates thereof remains after storage at 2° C. to 8° C. for preferably about 3 months, more preferably about 6 months, and still more preferably about 12 months.

A preferred embodiment of the invention is, in which the tetracycline antibiotic or its pharmaceutically acceptable salts or hydrates thereof is solubilised in the protic solvent.

Another preferred embodiment of the invention is, in which the tetracycline antibiotic or its pharmaceutically acceptable salts or hydrates thereof is suspended in the protic solvent.

According to the present invention, the tetracycline antibiotic is preferably employed in an amount ranging from about 0.00001% to about 25%, more preferably in an amount ranging from about 0.0025% to about 6%, and most preferably in an amount ranging from about 1% to about 5%, by weight of the formulation.

In a particularly preferred embodiment of the invention, the formulation comprises an ointment, e.g., a semisolid preparation comprising petrolatum or other petroleum derivatives.

In one aspect, the specific ointment base provides for desired characteristics, e.g., emolliency. As with other carriers or vehicles, an ointment base can be inert, stable, nonirritating and nonsensitizing. There are five classes or types of ointment bases including oleaginous bases, absorption bases, water in oil emulsion bases, oil in water emulsion bases and water soluble or water miscible bases which are differentiated on the basis of their physical composition. All of them can be made as water washable or non water washable.

Oleaginous bases are anhydrous hydrophobic and occlusive. Examples include vegetable oils, fats obtained from animals, and semisolid hydrocarbons obtained from petroleum, White

Petrolatum, White Ointment, hard paraffin, soft paraffin, microcrystalline wax and ceresine, vegetable oil, animal fat. Absorption bases are anhydrous hydrophilic and occlusive and examples include Hydrophilic Petrolatum, Anhydrous Lanolin, Aquabase™, Aquaphor®, Polysorb. Water in oil emulsion bases are hydrous hydrophilic with easy spreadability and wherein examples include Cold Cream type, Hydrous Lanolin, Rose Water Ointment, Hydrocream™, Eucerin®, Nivea®. Oil in water emulsion bases are hydrous hydrophilic and non-occlusive include examples hydrophilic Ointment, Dermabase™, Velvachol®, Unibase, cetyl alcohol, glyceryl monostearate, lanolin and stearic acid. Water soluble or water miscible bases are hydrous/anhydrous and non-occlusive which includes examples of PEG Ointment, Polybase, Macrogols.

In a particularly preferred embodiment of the invention, the formulation comprises creams, e.g., viscous liquids or semisolid emulsions, either oil-in-water or water-in-oil. Cream bases can be water-washable, and can contain an oil phase, an emulsifier and an aqueous phase. The oil phase can be petrolatum or a fatty alcohol such as cetyl or stearyl alcohol. The aqueous phase can exceed the oil phase in volume and can contain a humectant. The emulsifier in a cream formulation can comprise a nonionic, anionic, cationic or amphoteric surfactant.

In a preferred embodiment of the present invention, the formulation is suitable for external administration to the skin.

The term “pharmaceutically acceptable bases” as employed herein denotes any known or suitable pharmaceutical excipients which will produce a formulation which permits topical application and are nontoxic and non- sensitizing and compatible with the sap or exudates.

Pharmaceutically acceptable bases suitable for use in the topical formulation of the invention are selected from the group consisting of caprylic/capric triglycerides, castor oil, ceteareth-20, ceteareth-30, cetearyl alcohol, ceteth 20, cetostearyl alcohol, cetyl alcohol, cetyl stearyl alcohol, cocoa butter, diisopropyl adipate, glycerin, gyceryl monooleate, glyceryl monostearate, glyceryl stearate, isopropyl myristate, isopropyl palmitate, lanolin, lanolin alcohol, hydrogenated lanolin, liquid paraffins, linoleic acid, mineral oil, oleic acid, white petrolatum, polyethylene glycol, polyoxyethylene glycol fatty alcohol ethers, polyoxypropylene 15-stearyl ether, propylene glycol stearate, squalane, steareth-2 or -100, stearic acid, stearyl alcohol and urea and mixtures thereof.

Since it is known that tetracyclines may increase skin sensitivity to sunlight, the formulation may additionally contain a sunscreen, which offers the advantage of blocking sunlight to avoid this undesirable side effect.

Sunscreen agents suitable for use in the topical formulation of the invention are selected from the group consisting of avobenzone, octocrylene, octyl methoxycinnamate, octinoxate, aluminim starch octenyl succinate, zinc oxide, PABA, glyceryl PABA, padimate O, roxadimate, dioxybenzone, oxybenzone, sulisonbenzone, homosalate, ethylhexyl salicylate and Trolamine salicylate.

The base may further include one or more optional ingredients such as antioxidants, pH modifiers, preservatives, moisturizers, thickening agents, opacifiers, skin penetrants and emulsifiers (surfactants).

Antioxidants suitable for use in the topical formulation of the invention are selected from the group consisting of butylated hydroxyl toluene, butylated hydroxy anisole, sodium formaldehyde sulphoxylate, sodium sulphite, sodium metabisulphite, sodium formaldehyde sulphoxylate, ascorbic acid, methyl paraben, propyl paraben and propyl gallate.

pH modifiers suitable for use in the topical formulation of the invention are selected from the group consisting of sodium hydroxide, triethanolamine, diisopropanolamine, hydrochloric acid and combinations thereof.

Preservatives suitable for use in the topical formulation of the invention are selected from the group consisting of parahydroxybenzoate esters, sorbic acid and its salts, boric acid and borate salts and phenolics.

Moisturizers suitable for use in the topical formulation of the invention are selected from the group consisting of cetyl alcohol, or silicone-derived ingredients, such as cyclomethicone, hexylene glycol, petrolatum, Natural squalene, cocoa butter and glycerin.

Thickening, stiffening or suspending agents suitable for use in the topical formulation of the invention are selected from the group consisting of aluminum stearate, beeswax, synthetic beeswax, carbomer 934, carbomer 934P, carbomer 940, cetostearyl alcohol, cetyl alcohol, cetyl esters wax, dextrin, glyceryl monostearate, hydroxypropyl cellulose, kaolin, paraffin, petrolatum, polyethylene, poly propylene glycol stearate, starch, stearyl alcohol, wax, white wax, xanthan gum, and bentonite.

Opacifiers suitable for use in the topical formulation of the invention are selected from the group consisting of titanium dioxide, zinc oxide, and magnesium stearate.

Skin penetrants suitable for use in the topical formulation of the invention are selected from the group consisting of soya lecithin, phosphatidyl choline, propylene glycol, derivatized propylene glycol, diethylene glycol monomethylether, diethylene glycol monoethyl ether, dimethyl isosorbide, propylene glycol monolaurate, tri-block polyethers, isopropyl myristate, stearic acid, dimethyl sulfoxide, poloxamer 407, and combinations thereof.

Emulsifiers suitable for use in the topical formulation of the invention are selected from the group consisting of aluminum starch octenylsuccinate, ammonium hydroxide, amphoteric-9, beeswax, synthetic beeswax, carbomer 934, carbomer 934P, carbomer 940, ceteareth-20, ceteareth-30, cetearyl alcohol, ceteth 20, cetyl alcohol, cholesterol, cyclomethicone, diglycerides, dimethicone (e.g., dimethicone 350), disodium monooleamidosulfosuccinate, NF emulsifying wax, fatty acid pentaerythritol ester, glycerides, glyceryl monooleate, glyceryl monostearate, lanolin, lanolin alcohol, hydrogenated lanolin, magnesium stearate, mineral oil, monoglycerides, polyethylene glycol, PEG 100 stearate, polyethylene glycol 6000 distearate, polyethylene glycol 1000 monocetyl ether, polyethylene glycol monostearate, polyethylene glycol 400 monostearate, polyoxyethylene glycol fatty alcohol ethers, polyoxyl 20 cetostearyl ether, polyoxyl 40 stearate, polysorbate 20, polysorbate 40, polysorbate 60, polysorbate 80, polysorbates, PPG-26 oleate, propylene glycol stearate, quaternium-15, simethicone, sodium laureth sulfate, sodium lauryl sulfate, sorbitan esters, sorbitan monolaurate, sorbitan monooleate, sorbitan monopalmitate, sorbitan monostearate, sorbitan palmitate, sorbitan sesquioleate, steareth-2, steareth-100, stearic acid, stearyl alcohol, triethanolamine, trolamine, polymeric emulsifiers like Pemulen TR1 and TR2 and Promulgen G.

Preferably, the topical formulation contains calcium ions, suitably added as calcium chloride, to compensate for a calcium defect which appears to be present in vitiliginous skin.

According to a preferred embodiment of the present invention, there is provided a topical formulation comprising: (a) 0.01-5% by weight of minocycline or a physiologically acceptable salt thereof; (b) 1-15% by weight of a emulsifiers; (c) 1-50% by weight of a base; and (d) 0.01-0.5% by weight of a preservative (e) 0.01 to 2% by weight of antioxidants (t) and the weight is made to 100% by water.

According to a preferred embodiment of the present invention, there is provided a topical formulation comprising: (a) 0.01-5% by weight of minocycline or a physiologically acceptable salt thereof; (b) 1-15% by weight of a emulsifiers; (c) 1-50% by weight of a base; and (d) 0.01-0.5% by weight of a preservative (e) 0.01 to 2% by weight of an antioxidants (f) 0.1-10% by weight of sunscreen agents (g) and the weight is made to 100% by water.

The following examples are offered to illustrate, but not limit the claimed invention.

### EXAMPLE I

| Sr. No | Ingredient | % |
|---|---|---|
| 1 | Minocycline HCl | 1 to 5 |
| 2 | Cyclomethicone | 3 to 13 |
| 3 | Natural Squalane | 2 to 5 |
| 4 | Cetostearyl alcohol | 10 to 25 |
| 5 | Ceteareth 20 | 3 to 15 |
| 6 | Steareth 2 | 0.5 to 3 |
| 7 | Methyl paraben | 0.1 to 0.5 |
| 8 | Propyl paraben | 0.01 to 0.3 |
| 9 | Calcium Chloride | 0.05 to 2 |
| 10 | Butylated hydroxyl toluene | 0.01 to 2 |
| 11 | Purified Water | q.s. to 100 |
| | TOTAL | 100 |

Manufacturing Procedure:

i. Take cyclomethicone, natural Squalane, cetostearyl alcohol, ceteareth 20, steareth 2, butylated hydroxyl toluene and propyl paraben in a vessel. Heat to 65° C. to 70° C. and melt completely.

ii. Add Minocycline HCl into mixture prepared in step 1 under stirring and disperse well.

iii. Take purified water and add methyl paraben and calcium chloride under stirring and dissolve completely.

iv. Add solution prepared in step iii to step ii under stirring and stir for 30 minutes.

v. Cool to room temperature under stirring.

### EXAMPLE II

| Sr. No | Ingredient | % |
|---|---|---|
| 1 | Minocycline HCl | 1 to 5 |
| 2 | Cyclomethicone | 3 to 13 |
| 3 | Natural Squalane | 2 to 5 |
| 4 | Cetostearyl alcohol | 10 to 25 |
| 5 | Ceteareth 20 | 3 to 15 |
| 6 | Steareth 2 | 0.5 to 3 |
| 7 | Methyl paraben | 0.1 to 0.5 |
| 8 | Propyl paraben | 0.01 to 0.3 |
| 9 | Calcium Chloride | 0.05 to 2 |
| 10 | Butylated hydroxyl toluene | 0.01 to 2 |
| 11 | Sodium hydroxide solution | To adjust pH 7 |
| | (10%) | |
| 12 | Purified Water | q.s. to 100 |
| | TOTAL | 100 |

Manufacturing Procedure:

i. Take cyclomethicone, natural Squalane, cetostearyl alcohol, ceteareth 20, steareth 2, butylated hydroxyl toluene and propyl paraben in a vessel. Heat to 65° C. to 70° C. and melt completely.

ii. Take part quantity of purified water and add methyl paraben and calcium chloride under stirring and dissolve completely.

iii. Take remaining quantity of purified water and add Minocycline HCl into it under stirring. Heat it to get a clear solution. Then add sodium hydroxide solution (10%) and adjust the pH 7.

iv. Add solution prepared in step ii to step iii and mix well under stirring. Heat and maintain temperature at 65° C. to 70° C.

v. Add solution prepared in step iv to the mixture prepared in step i under stirring and stir for 30 minutes.

vi. Cool to room temperature under stirring.

### EXAMPLE III

| Sr. No | Ingredient | % |
|---|---|---|
| 1 | Minocycline HCl | 1 to 5 |
| 2 | Cyclomethicone | 3 to 13 |
| 3 | Natural Squalane | 2 to 5 |
| 4 | Cetostearyl alcohol | 10 to 25 |
| 5 | Ceteareth 20 | 3 to 15 |
| 6 | Steareth 2 | 0.5 to 3 |
| 7 | Avobenzone | 0.1 to 5 |
| 8 | Methyl paraben | 0.1 to 0.5 |
| 9 | Propyl paraben | 0.01 to 0.3 |
| 10 | Calcium Chloride | 0.05 to 2 |
| 11 | Butylated hydroxyl | 0.01 to 2 |
| | toluene | |
| 12 | Purified Water | q.s. to 100 |
| | TOTAL | 100 |

Manufacturing Procedure:

i. Take cyclomethicone, natural Squalane, cetostearyl alcohol, ceteareth 20, steareth 2, butylated hydroxyl toluene, avobenzone and propyl paraben in a vessel. Heat to 65° C. to 70° C. and melt completely.

ii. Add Minocycline HCl into mixture prepared in step i under stirring and disperse well.

iii. Take purified water and add methyl paraben, calcium chloride and under stirring and dissolve completely.

iv. Add solution prepared in step iii to step ii under stirring and stir for 30 minutes.

v. Cool to room temperature under stirring.

### EXAMPLE IV

| Sr. No | Ingredient | % |
|---|---|---|
| 1 | Minocycline HCl | 1 to 5 |
| 2 | Cyclomethicone | 3 to 13 |
| 3 | Natural Squalane | 2 to 5 |
| 4 | Cetostearyl alcohol | 10 to 25 |
| 5 | Ceteareth 20 | 3 to 15 |
| 6 | Steareth 2 | 0.5 to 3 |
| 7 | Avobenzone | 0.1 to 5 |
| 8 | Methyl paraben | 0.1 to 0.5 |
| 9 | Propyl paraben | 0.01 to 0.3 |
| 10 | Calcium Chloride | 0.05 to 2 |
| 11 | Butylated hydroxyl toluene | 0.01 to 2 |
| 12 | Sodium hydroxide solution | To adjust pH 7 |
| | (10%) | |
| 13 | Purified Water | q.s. to 100 |
| | TOTAL | 100 |

Manufacturing Procedure:

i. Take cyclomethicone, natural Squalane, cetostearyl alcohol, ceteareth 20, steareth 2, butylated hydroxyl toluene, avobenzone and propyl paraben in a vessel. Heat to 65° C. to 70° C. and melt completely.

ii. Take part quantity of purified water and add methyl paraben, calcium chloride and under stirring and dissolve completely.

iii. Take remaining quantity of purified water and add Minocycline HCl into it under stirring. Heat it to get a clear solution. Then add sodium hydroxide solution (10%) and adjust the pH 7.

iv. Add solution prepared in step ii to step iii and mix well under stirring. Heat and maintain temperature at 65° C. to 70° C.

v. Add solution prepared in step 4 to the mixture prepared in step i under stirring and stir for 30 minutes.

vi. Cool to room temperature under stirring.

### EXAMPLE V

| Sr. No | Ingredient | % |
|---|---|---|
| 1 | Minocycline HCl | 1 to 5 |
| 2 | Hexylene glycol | 9 to 25 |
| 3 | Purified Water | 2 to 3 |
| 4 | Sodium hydroxide solution (10%) | To adjust pH 7 |
| 5 | Promulgen G (Stearyl alcohol | 5 to 15 |
| | and Ceteareth 20) | |
| 6 | Titanium dioxide | 0.5 to 2 |
| 7 | Aluminium starch octenyl succinate | 5 to 15 |
| 8 | White wax | 1 to 5 |
| 9 | White petrolatum | q.s to 100 |
| | TOTAL | 100 |

Manufacturing Procedure:

i. Take white petrolatum, white wax and promulgen g and heat to 70° to 75° C. and melt completely.

ii. Take hexylene glycol and add into it purified water and mix well. Add Minocycline HCl and mix well under stirring. Adjust pH of the solution to 7.

iii. Add the solution prepared in step ii to step i under stirring. Homogenize for 20 to 30 minutes at 70° C. to 75° C.

iv. After completion of homogenization add titanium dioxide and aluminium starch octenyl succinate under stirring and mix well. Homogenize for 10 minutes at 70° C. to 75° C.

v. Cool to room temperature under stirring.

### EXAMPLE VI

| Sr. No | Ingredient | % |
|---|---|---|
| 1 | Minocycline HCl | 1 to 5 |
| 2 | Hexylene glycol | 9 to 25 |
| 3 | Purified Water | 2 to 3 |
| 4 | Sodium hydroxide solution (10%) | To adjust pH 7 |
| 5 | Promulgen G (Stearyl alcohol | 5 to 15 |
| | and Ceteareth 20) | |
| 6 | Titanium dioxide | 0.5 to 2 |
| 7 | Avobenzone | 0.1 to 5 |
| 8 | Aluminium starch octenyl succinate | 5 to 15 |
| 9 | White wax | 1 to 5 |
| 10 | White petrolatum | q.s to 100 |
| | TOTAL | 100 |

i. Take white petrolatum, white wax and promulgen g and avobenzone. Heat to 70° to 75° C. and melt completely.

ii. Take hexylene glycol and add into it purified water and mix well. Add Minocycline HCl and mix well under stirring. Adjust pH of the solution to 7.

iii. Add the solution prepared in step ii to step i under stirring. Homogenize for 20 to 30 minutes at 70° C. to 75° C.

iv. After completion of homogenization add titanium dioxide and aluminium starch octenyl succinate under stirring and mix well. Homogenize for 10 minutes at 70° C. to 75° C.

v. Cool to room temperature under stirring.

### EXAMPLE VII

| Sr. No | Ingredient | % |
|---|---|---|
| 1 | Minocycline HCl | 1 to 5 |
| 2 | Dimethyl Isosorbide and/or | 10 to 25 |
| | Diethylene glycol monoethyl ether | |
| 3 | Glyceryl stearate and PEG 100 stearate | 1 to 10 |
| 4 | Glyceryl monostearate | 1 to 10 |
| 5 | Carbomer 940 | 0.1 to 1.5 |
| 6 | Diisopropanolamine (5 to 20% solution) or | To adjust pH 5 to 7 |
| | Triethanolamine (5 to 20% solution) | |
| 7 | Calcium Chloride | 0.05 to 2 |
| 8 | Butylated hydroxyl toluene | 0.01 to 2 |
| 9 | Propylene Glycol | q.s to 100 |
| | TOTAL | 100 |

Manufacturing Procedure:

i. Take part quantity of propylene glycol, and add under stirring carbomer 940 and disperse completely. Heat and maintain temperature at 60° C. to 65° C.

ii. Take part quantity of propylene glycol and add Minocycline into it under stirring and heat to 60° C. to 65° C. and dissolve completely.

iii. Take part quantity of propylene glycol and dimethyl Isosorbide and/or Diethylene glycol monoethyl ether and add calcium chloride into it under stirring. Mix well to dissolve completely.

iv. Add the mixture prepared in step iii to i and mix well. Heat and maintain temperature at 60° C. to 65° C.

v. Take glyceryl stearate and peg 100 stearate, glyceryl monostearate and BHT. Heat to 60° C. to 65 ° C. and melt completely. Maintain temperature at 60° C. to 65° C.

vi. Add mixture prepared in step v to the mixture prepared in step iv and homogenize for 30 minutes.

15 vii. Add the diisopropanolamine solution (5 to 20% solution) or triethanolamine solution (5 to 20% solution) to pH 5 to 7.

viii. Cool to temperature 35° C. to 40° C. Add the solution prepared in step ii and mix well.

ix. Cool to room temperature under stirring.

### EXAMPLE VIII

| Sr. No | Ingredient | % |
|---|---|---|
| 1 | Minocycline HCl | 1 to 5 |
| 2 | Dimethyl Isosorbide and/or | 10 to 25 |
| | Diethylene glycol monoethyl ether | |
| 3 | Glyceryl stearate and PEG 100 stearate | 1 to 10 |
| 4 | Glyceryl monostearate | 1 to 10 |
| 5 | Avobenzone | 0.1 to 5 |
| 6 | Carbomer 940 | 0.1 to 1.5 |
| 7 | Diisopropanolamine (5 to 20% solution) or | To adjust pH 5 to 7 |
| | Triethanolamine (5 to 20% solution) | |
| 8 | Calcium Chloride | 0.05 to 2 |
| 9 | Butylated hydroxyl toluene | 0.01 to 2 |
| 10 | Propylene Glycol | q.s to 100 |
| | TOTAL | 100 |

Manufacturing Procedure:

i. Take part quantity of propylene glycol, and add under stirring carbomer 940 and disperse completely. Heat and maintain temperature at 60° C. to 65° C.

ii. Take part quantity of propylene glycol and add Minocycline into it under stirring and heat to 60° C. to 65° C. and dissolve completely.

iii. Take part quantity of propylene glycol and dimethyl Isosorbide and/or Diethylene glycol monoethyl ether and add calcium chloride into it under stirring. Mix well to dissolve completely.

iv. Add the mixture prepared in step iii to i and mix well. Heat and maintain temperature at 60° C. to 65° C.

v. Take glyceryl stearate, PEG 100 stearate, glyceryl monostearate, avobenzone and BHT. Heat to 60° C. to 65 ° C. and melt completely. Maintain temperature at 60° C. to 65° C.

vi. Add mixture prepared in step v to the mixture prepared in step iv and homogenize for 30 minutes.

vii. Add the diisopropanolamine solution (5 to 20% solution) or triethanolamine solution (5 to 20% solution) to pH 5 to 7.

viii. Cool to temperature 35° C. to 40° C. Add the solution prepared in step ii and mix well.

ix. Cool to room temperature under stirring.

### EXAMPLE IX

| Sr. No | Ingredient | % |
|---|---|---|
| 1 | Minocycline HCl | 1 to 5 |
| 2 | Dimethyl Isosorbide and/or | 10 to 25 |
| | Diethylene glycol monoethyl ether | |
| 3 | Glyceryl stearate and PEG 100 stearate | 1 to 10 |
| 4 | Glyceryl monostearate | 1 to 10 |
| 5 | Avobenzone | 0.1 to 5 |
| 6 | Titanium dioxide | 0.1 to 5 |
| 7 | Hydroxypropyl cellulose | 0.1 to 4 |
| 8 | Calcium Chloride | 0.05 to 2 |
| 9 | Butylated hydroxyl toluene | 0.01 to 2 |
| 10 | Propylene Glycol | q.s to 100 |
| | TOTAL | 100 |

Manufacturing Procedure:

i. Take part quantity of propylene glycol, dimethyl Isosorbide and/or Diethylene glycol monoethyl ether, calcium chloride and BHT into it under stirring. Mix well to dissolve completely. Heat to 60° C. to 65° C. and maintain temperature.

ii. Add in step i Minocycline into it under stirring and dissolve completely.

iii. Add in step ii under stirring hydroxypropyl cellulose and disperse completely. Heat and maintain temperature at 60° C. to 65° C.

iv. Take glyceryl stearate, peg 100 stearate, glyceryl monostearate and avobenzone. Heat to 60° C. to 65 ° C. and melt completely. Maintain temperature at 60° C. to 65° C.

v. Add the mixture prepared in step IV to the mixture prepared in step iii and homogenize for 30 minutes.

vi. Add titanium dioxide and mix well.

vii. Cool to room temperature under stirring.

1. A topical formulation comprising at least one tetracycline antibiotic for the treatment of acne, wherein the tetracycline antibiotic is in stabilized form.

2. The topical formulation according to claim 1, wherein the tetracycline antibiotic is present in a free base form, a hydrate form, a salt form or a complex form.

3. The topical formulation according to claim 2, wherein the tetracycline antibiotic is selected from the group consisting of tetracycline, minocycline, doxycycline, oxytetracycline, chlortetracycline, demeclocycline, methacycline, tigecycline and combinations thereof.

4. The topical formulation according to claim 1, wherein the concentration of tetracycline antibiotic is from about 0.00001% w/w to about 25% w/w.

5. The topical formulation according to claim 3, wherein the formulation comprises minocycline.

6. The topical formulation according to claim 1, wherein the topical formulation is selected from the group consisting of ointments, lotions, pastes, creams, jellies, sprays, aerosols, gels, emulsions, suspensions and bath oils.

7. A topical formulation comprising at least one tetracycline antibiotic for the treatment of acne, wherein the tetracycline antibiotic is in solubilized form.

8. The topical formulation according to claim 7, wherein the tetracycline antibiotic is solubilized in a protic solvent.

17. A topical formulation comprising at least one tetracycline antibiotic for the treatment of acne, wherein the tetracycline antibiotic is in suspended form.

18. The topical formulation according to claim 17, wherein the tetracycline antibiotic is suspended in the protic solvent.

19. The topical formulation according to claim 18, wherein the protic solvent is selected from the group consisting of water, alcohols, glycols, acids and bases.

20. The topical formulation according to claim 17, wherein the tetracycline antibiotic is present in a free base form, a hydrate form, a salt form or a complex form.

21. The topical formulation according to claim 20, wherein the tetracycline antibiotic is selected from the group consisting of tetracycline, minocycline, doxycycline, oxytetracycline, chlortetracycline, demeclocycline, methacycline, tigecycline and combinations thereof.

22. The topical formulation according to claim 17, wherein the concentration of tetracycline antibiotic is from about 0.00001% w/w to about 25% w/w.

23. The topical formulation according to claim 21, wherein the formulation comprises minocycline.

24. The topical formulation according to claim 17, further comprising a sunscreen agent.

25. The topical formulation according to claim 24, wherein the sunscreen agent is selected from the group consisting of avobenzone, octocrylene, octyl methoxycinnamate, octinoxate, aluminim starch octenyl succinate, zinc oxide, PABA, glyceryl PABA, padimate O, roxadimate, dioxybenzone, oxybenzone, sulisonbenzone, homosalate, ethylhexyl salicylate and Trolamine salicylate.

26. The topical formulation according to claim 17, wherein the topical formulation is selected from the group consisting of ointments, lotions, pastes, creams, jellies, sprays, aerosols, gels, emulsions, suspensions and bath oils.
